(19) United States
(12) Patent Application Publication
(10) Pub. No.: US 2006/0204575 A1
(43) Pub. Date: Sep. 14, 2006

(54) Amphetamine formulations

(76) Inventors: Hengsheng Feng, Edison, NJ (US); Grant Heinicke, Rockaway, NJ (US)

Correspondence Address: CANTOR COLBURN, LLP, 55 GRIFFIN ROAD SOUTH, BLOOMFIELD, CT, 06002, US

(21) Appl. No.: 11/078,982

(22) Filed: Mar. 11, 2005

(51) Int. Cl. A61K 31/137; A61K 9/26

(52) U.S. Cl. 424/469; 514/649

(57) ABSTRACT

Amphetamine formulations comprise a plurality of particles comprising two or more pharmaceutically active amphetamine salts, wherein the dosage form, when administered under fed conditions provides a reduced variability in bioavailability as compared to the dosage form administered under fasted conditions.

### BACKGROUND

Continued development and improvement of sustained-release pharmaceutical compositions has long been a focus in the field of pharmaceutical formulations. Advantages of a sustained-release composition include increased convenience of administration, and also a more consistent therapeutic effect throughout a desired period of time. Among the methods of measurement of the therapeutic effects of particular dosage forms, bioavailability and bioequivalence are the most commonly referenced parameters that should be determined under both fasted and fed conditions. Suitable criteria include, but are not limited to, AUC (total exposure, or area under the concentration-time curve), Cₘₐₓ (peak exposure), Tₘₐₓ (time to maximum blood plasma concentration), t_{lag} (lag-time); terminal elimination half-life, and the like. A significant challenge in the formulation of a therapeutically effective dosage form is to reduce or minimize the variations in all or the most of the above referenced criteria under both fasted and fed conditions.

Attention-Deficit/Hyperactivity Disorder (ADHD) is a behavior disorder characterized by problems with control of attention and hyperactivity-impulsivity. A large population of patients with ADHD symptoms are children, among whom attention deficit or inattention becomes apparent upon entry to elementary school. It is well recognized among those skilled in the art that a dosage form with a reduced food effect is more desirable for this particular population.

Amphetamines are non-catecholamine, sympathominetic amines with central nervous system stimulant activity, which have been widely utilized in treating narcolepsy and ADHD. The amphetamine salts generally include the neutral sulfate salts of dextroamphetamine and amphetamine, the dextro isomer of amphetamine saccharate and d,l-amphetamine aspartate. Currently marketed oral products to treat narcolepsy and/or ADHD include both immediate-release and modified-release forms. The Dexedrine® product is marketed as a sustained-release formulation with dextroamphetamine sulfate as the only active ingredient. Adderall XR™ is a once daily sustained-release, single-entity dosage form containing four amphetamine salts which is indicated for treatment of ADHD in children from 3 to 10 years of age. A food effect of Adderall XR™ was observed during its clinical studies. For example, in a clinical study with human subjects, after oral administration of a 30-mg strength Adderall XR™, Tₘₐₓ was prolonged from 4.9 hours under fasted conditions to 8.2 hours under fed conditions and Cₘₐₓ varied from 39.9 ng/mL under fed conditions to 47.9 ng/mL under fasted conditions.

A multiple pulsed-dosage drug delivery system for pharmaceutically active amphetamine salts is described in U.S. Pat. Nos. 6,322,819 and 6,287,599. This system comprises an immediate-release portion releasing drug in stomach and an enteric pulsed-release portion releasing drug in the small intestine, which a person skilled in the art would recognize an inherent food effect.

U.S. Pat. No. 6,384,020 discloses a rapid immediate-release oral dosage form in delivering amphetamines and their salts. This dosage form solves particular process problems and has an adequate stability profile. However, this reference does not teach how to make a sustained-release formulation.

The present invention addresses the need for additional amphetamine dosage forms, particularly extended release dosage forms.

### SUMMARY

This invention includes a sustained-release pharmaceutical composition for administration of two or more amphetamine salts to a patient, wherein the composition exhibits a reduced variability in bioavailability under fed versus fasted conditions. In one embodiment, the variation in Tₘₐₓ under fed versus fasted conditions is less than or equal to about a 30% increase or decrease in time from administration and the variation in Cₘₐₓ under fed versus fasted conditions is less than or equal to about a 15% increase or decrease.

The invention also includes a sustained-release pharmaceutical dosage form for administration of two or more amphetamine salts to a human comprising a plurality of particles. In one embodiment, the particles comprise a first population of immediate-release particles; and a second population of sustained-release particles comprising one or more sustained-release polymers, wherein the sustained-release particles comprise no enteric release polymers.

In another embodiment, this invention includes a method of treating a human in need of two or more amphetamine salts comprising administering the foregoing dosage form.

These and other embodiments, advantages and features of the present invention become clear when detailed description and examples are provided in subsequent sections.

### BRIEF DESCRIPTION OF THE DRAWINGS

FIG. 1 shows dissolution profiles for sustained-release pellets having different sustained-release coating weights.

FIG. 2 shows the dissolution profile for an exemplary dosage form.

FIG. 3 shows the dissolution profile of another exemplary dosage form.

FIG. 4 shows the mean dextroamphetamine concentration versus time for an exemplary inventive dosage form compared to Adderall XR™ when dosed under fasted conditions.

FIG. 5 shows the mean levoamphetamine concentration versus time for an exemplary inventive dosage form compared to Adderall XR™ when dosed under fasted conditions.

FIG. 6 shows the mean dextroamphetamine concentration versus time for an exemplary inventive dosage form compared to Adderall XR™ when dosed under fed conditions.

FIG. 7 shows the mean levoamphetamine concentration versus time for an exemplary inventive dosage form compared to Adderall XR™ when dosed under fed conditions.

### DETAILED DESCRIPTION

The term “active agent” is meant to include solvates (including hydrates) of the free compound or salt, crystalline and non-crystalline forms, as well as various polymorphs. Unless otherwise specified, the term “active agent” is used herein to indicate a pharmaceutically active amphetamine or a pharmaceutically acceptable salt thereof. For example, an active agent can include all optical isomers of amphetamines and all pharmaceutically acceptable salts thereof either alone or in combination.

“Pharmaceutically acceptable salts” includes derivatives of amphetamines, wherein the amphetamine is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the amphetamine formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, aspartate, asparginate, glutamate, succharate and the like; and combinations comprising one or more of the foregoing salts.

By “oral dosage form” is meant to include a unit dosage form prescribed or intended for oral administration. An oral dosage form may or may not comprise a plurality of subunits such as, for example, microcapsules or microtablets, packaged for administration in a single dose.

By “releasable form” is meant to include immediate-release, controlled-release, and sustained-release forms. Certain release forms can be characterized by their dissolution profile. Dissolution profile as used herein, means a plot of the cumulative amount of active ingredient released as a function of time.

By “immediate-release”, it is meant a conventional or non-modified release in which greater then or equal to about 75% of the active agent is released within two hours of administration, preferably within one hour of administration.

“Sustained-release” or “extended-release” include the release of the active agent for an extended period of time such that blood (e.g., plasma) levels are maintained within a therapeutic range, but below toxic levels, for at least about 4 hours to about 24 hours at steady state. The term steady-state means that a plasma level for a given active agent has been achieved and which is maintained with subsequent doses of the drug at a level which is at or above the minimum effective therapeutic level and is below the minimum toxic plasma level for a given active agent. Sustained-release as used herein includes a consistent level of release as well as periodic release so long as the release occurs over an extended period of time.

In one embodiment, the disclosed dosage forms, when administered under fed conditions, provide less than or equal to about a 30% increase or decrease in time to maximum blood plasma concentration of pharmaceutically active amphetamine salt (Tₘₐₓ) as compared to the dosage form administered under fasted conditions. In other embodiments, the dosage forms, when administered under fed conditions, provide less than or equal to about a 20%, about a 15%, or about a 10% increase or decrease in time to maximum blood plasma concentration of pharmaceutically active amphetamine salt (Tₘₐₓ) as compared to the dosage form administered in fasted conditions.

In another embodiment, the dosage forms, when administered under fed conditions, provide less than or equal to about a 15% increase or decrease in bioavailability measured as Cₘₐₓ as compared to the dosage forms administered under fasted conditions. In other embodiments, the dosage forms, when administered under fed conditions, provide less than or equal to about a 12%, or about a 10% increase or decrease in Cₘₐₓ as compared to the dosage forms administered in fasted conditions. In another embodiment, the dosage forms may have a reduced inter- and/or intra-patient variability in bioavailability measured as Cₘₐₓ.

In one embodiment, the dosage forms have a confidence interval of about 80% to 125% of the log-transformed parameter in comparison to the same parameter of ADDERAL XR™, wherein the parameter is selected from the group consisting of Cₘₐₓ, AUCₗₐₛₜ and AUC_{0-INF}.

In one embodiment, an amphetamine formulation comprises one or more populations of particles comprising two or more pharmaceutically active amphetamine salts. The term particles as used herein encompasses pellets, granules and the like. At least a portion of the particles may provide a sustained-release of the amphetamine salt upon ingestion by a mammal. The amphetamine salt may comprise dextroamphetamine sulfate, dextroamphetamine saccharate, amphetamine aspartate, amphetamine sulfate, or a combination comprising two or more of the foregoing amphetamine salts.

A sustained-released pellet comprises two or more pharmaceutically active amphetamine salts and a sustained-release agent. Suitable sustained-release agents include hydrophilic sustained-release agents, hydrophobic sustained-release agents, and combinations thereof. Suitable hydrophilic polymers include, for example, hydroxypropylmethyl cellulose, methylcellulose, hydroxypropyl cellulose, sodium carboxymethyl cellulose, and combinations comprising one or more of the foregoing hydrophilic polymers. Suitable hydrophobic polymers include, for example, ethyl cellulose, cellulose acetate, polyvinyl alcohol-maleic anhydride copolymers, acrylic acid polymers, and combinations comprising one or more of the foregoing hydrophobic polymers. The sustained-release agent may, for example, be in the form of a binder and/or a sustained-release coating.

One embodiment of an amphetamine formulation comprises a first population of immediate-release pellets, and a second population of sustained-release pellets, wherein the first population and the second population comprise two more pharmaceutically active amphetamine salts. In one embodiment, the amphetamine salts comprise dextroamphetamine sulfate, dextroamphetamine saccharate, amphetamine aspartate monohydrate, and amphetamine sulfate.

The amphetamine formulations include those based on pellets comprising an inert core having disposed thereon a coating composition comprising one or more pharmaceutically active amphetamine salts. Suitable inert cores include for example, sugar spheres, particulate microcrystalline cellulose, silicon dioxide spheres, wax beads such as prilled waxes, and combinations comprising one or more of the foregoing inert cores. In one embodiment, the core comprises non-pareil sugar seeds having an average size of about 25 to about 35 mesh (500 to 710 micrometers), or about 25 to about 30 mesh (600 to 710 micrometers), or about 30 to about 35 mesh (500-600 micrometers). The inert core comprises about 50 wt % to about 90 wt % of the pellets, specifically about 88.25 wt % of the pellets.

A mixture comprising two or more pharmaceutically active amphetamine salts and a binder is disposed on the inert core in an amount sufficient to provide a dosage form comprising about 1 to about 50 mg of pharmaceutically active amphetamine salt. The term mixture is meant to include both solutions and suspensions. A suitable binder would adhere particles of the selected composition to a core, but would not create such adhesive effects wherein particles of the selected composition adhere to one another. Suitable binders include, for example, hydroxypropyl cellulose, hydroxypropyl methylcellulose, methylcellulose, hydroxyethyl cellulose, polyvinylpyrrolidone, polyvinylpyrrolidone/vinyl acetate copolymers, and combinations comprising one or more of the foregoing binders. In on embodiment, the binder comprises hydroxypropyl cellulose. The immediate-release pellets comprise about 0.001 wt % to about 4 wt % of the binder, specifically about 1.75 wt %.

The coating mixture comprises two or more pharmaceutically active amphetamine salts. Suitable amphetamine salts include chemical and chiral derivatives. In one embodiment of this invention a mixture of amphetamine salts is employed, i.e., d-amphetamine sulfate, d,1-amphetamine aspartate, d-amphetamine saccharate, and d,1-amphetamine sulfate. The mixture of amphetamine salts may be in a 1:1:1:1 ratio. The immediate-release pellets comprise about 5 wt % to about 15 wt % of the pharmaceutically active amphetamine salts, specifically about 5wt % to about 12 wt %.

The coating mixture also comprises a dispersing agent. In one embodiment, the binder is soluble in the dispersing agent to allow for easier processing and spraying of the drug onto the inert core. Examples of suitable dispersing agents include water, alcohol, isopropyl alcohol, acetone, alcohol USP (95% ethanol, 5% water), SDA-3A alcohol (denatured alcohol with methanol as a denaturant), and combinations comprising one or more of the foregoing dispersing agents.

The coating mixture may comprise other additives such as, for example, disintegration agents, fillers, surfactants, solubilizers, stabilizers, and combinations comprising one or more of the forgoing additives. Suitable disintegration agents include, for example, corn starch, pregelatinized starch, cross-linked carboxymethylcellulose, sodium starch glycolate, cross-linked polyvinylpyrrolidone, and combinations comprising one or more of the foregoing disintegration agents. Suitable fillers include, for example, lactose, calcium carbonate, calcium phosphate, calcium sulfate, microcrystalline cellulose, dextran, starches, sucrose, xylitol, lactitol, mannitol, sorbitol, sodium chloride, polyethylene glycol, and combinations comprising one or more of the foregoing fillers. Suitable surfactants include, for example, sodium lauryl sulfate, sorbitan monooleate, polyoxyethylene sorbitan monooleate, bile salts, glyceryl monostearate, and combinations comprising one or more of the foregoing surfactants. Suitable solubilizers include, for example, citric acid, succinic acid, fumaric acid, malic acid, tartaric acid, maleic acid, glutaric acid, sodium bicarbonate, sodium carbonates, and combinations comprising one or more of the foregoing solubilizers. Suitable stabilizers include, for example, antioxidation agents, buffers, acids, and combinations comprising one or more of the foregoing stabilizers.

Coated inert cores may be formed by dissolving the binder in the dispersing agent to form a granulating solution, adding the two or more pharmaceutically active amphetamine salts to the granulating solution to form a granulating suspension, and disposing the granulating suspension onto the inert cores. Disposing the granulating suspension on the cores can be performed by, for example, use of a fluid bed processor. Coating can be performed with a top spray process or a bottom spray process. Alternative core processing methods include, for example, granulation and extrusion-spheronization.

A protective coating layer may be disposed on the inert core before and/or after coating of the active agent. The inert core may be coated with a protective layer comprising a second binder (sub-coat) and/or an active agent-coated core may be coated with a protective layer comprising a third binder (outer-coat), by coating techniques such as pan coating or fluid bed coating using solutions of polymers in water or suitable organic solvents or by using aqueous polymer dispersions. The binder for the active agent-containing layer (i.e., the first binder) may be the same or different than the second and third binders. Exemplary materials for the protective coating layer include cellulose derivatives such as hydroxyethyl cellulose, hydroxypropyl cellulose, hydroxypropyl methylcellulose, polyvinylpyrrolidone, polyvinylpyrrolidone/vinyl acetate copolymer, ethyl cellulose aqueous dispersions, EUDRAGIT® RL 30D, OPADRY® and the like. The coating levels may be 0.5 wt % to 5 wt % of the weight of the pellets. In the case of an immediate-release pellet, a coating layer disposed over the API-coated core should not appreciably affect the immediate release properties of the pellet.

The coated active cores, or immediate-release pellets, may then be dried, for example, for about 0.25 to about 2 hours at a temperature of about 30° C. to about 60° C., allowed to cool to ambient temperature, and sieved through an appropriately sized mesh.

The immediate-release pellets and the sustained-release pellets may comprise the same amphetamine-coated inert core, or different amphetamine-coated inert-cores.

Sustained-release pellets include a core comprising a pharmaceutically active amphetamine salt further coated with an sustained-release coating composition. Suitable sustained-release pellets include an inert core, a first coating comprising a binder and a pharmaceutically active amphetamine salt disposed on the inert core, and a second coating comprising a sustained-release composition. The sustained-release pellets may comprise about 50 wt % to about 90 wt % of the inert core, about 0.001 wt % to about 4 wt % of the binder, about 5 wt % to about 15 wt % of the pharmaceutically active amphetamine salt, and about 10 wt % to about 40 wt % of the sustained-release polymer, all based on the total weight of the sustained-release pellets.

The sustained-release coating composition comprises a sustained-release polymer and no enteric polymers. As used herein, a sustained-release polymer is a polymer that creates a lag-time in drug release in a substantially pH-independent manner. Sustained-release polymers are distinguished from enteric polymers, which typically have a pH-sensitive dissolution.

Suitable polymers for the sustained-release coating include, for example, cellulose acetate, cellulose acetate butyrate, ethyl cellulose acetate propionate, ethyl cellulose, fatty acids and their esters, waxes, zein, copolymers of acrylates and methacrylates with quaternary ammonium groups, cellulose acetate latexes and combinations comprising one or more of the foregoing sustained-release polymers. Ethyl cellulose may be in the form of an aqueous dispersion

The sustained-release polymers may comprise a minor proportion of a first polymer that is permeable to water and a major proportion of a polymer that is less permeable to water than the first polymer. The permeable first polymer may comprise a cationic polymer synthesized from acrylic and methacrylic acid esters with a low content of quaternary ammonium groups, for example, EUDRAGIT® RL manufactured by Rohm Pharma GmbH. The permeability of EUDRAGIT® RL is reported as independent of pH. A less permeable second polymer comprises another such cationic polymer, for example, EUDRAGIT® RS manufactured by Rohm Pharma GmbH. The permeability of EUDRAGIT® RS is reported to be also independent of pH. The ratio of the first polymer that is permeable to water to the second polymer that is less permeable to water may be about 80:20 to about 90:10.

The sustained-release polymer or polymers comprise about 10 wt % to about 40 wt % of the total weight of the sustained-release pellets, specifically about 10 wt % to about 30 wt % of the sustained-release pellets.

The sustained-release coating may optionally comprise up to about 10 wt % of a pore-forming agent to adjust the release characteristics of the coating. Suitable pore-forming agents include, for example, Opadry Clear YS-1-7006, polyethylene glycol, Eudragit L-100 which forms pores in ethylcellulose coats at pH 5.5 or greater, lactose, hydroxypropylmethylcellulose, hydroxypropylcellulose, a polycarbonate, and combinations comprising one or more of the foregoing pore-forming agents.

The sustained-release coating may also comprise up to about 4 wt % of a plasticizer based on the total pellet weight. Suitable plasticizers include, for example, a vegetable oil, for example castor oil, or glycerol; a glyceryl ester of a fatty acid, for example glyceryl triacetate or glyceryl monoricinoleate; dibutyl sebacate; triethyl citrate; acetyl triethyl citrate; tributyl citrate; acetyl tributyl citrate; diethyl phthalate; dimethyl phthalate; and combinations comprising one or more of the foregoing plasticizers. In one embodiment, the plasticizer comprises triethyl citrate.

The sustained-release coating layer may optionally include a lubricant, a wetting agent or a combination comprising one or more of the foregoing additives. Suitable wetting agents include, for example, sodium lauryl sulfate, acacia, benzalkonium chloride, cetomacrogol emulsifying wax, cetostearyl alcohol, cetyl alcohol, cholesterol, diethanolamine, docusate sodium, sodium stearate, emulsifying wax, glyceryl monostearate, hydroxypropyl cellulose, lanolin alcohols, lecithin, mineral oil, monoethanolamine, poloxamer, polyoxyethylene alkyl ethers, polyoxyethylene castor oil derivatives, polyoxyethylene sorbitan fatty acid esters, polyoxyethylene stearates, propylene glycol alginate, sorbitan esters, stearyl alcohol and triethanolamine, and combinations comprising one or more of the foregoing wetting agents. Suitable lubricants include, for example, talc, calcium stearate, colloidal silicon dioxide, glycerin, magnesium stearate, mineral oil, polyethylene glycol, and zinc stearate, aluminum stearate, and combinations comprising one or more of the foregoing lubricants.

The amphetamine-coated cores may be dusted with talc prior to application of the sustained-release coating. The sustained-release coating may be deposited by dissolving the sustained-release polymers and optional additional additives in a dispersing agent, and disposing the solution onto the API-coated cores. Disposing the sustained-release coating mixture onto the API-coated cores can be performed by, for example, use of a fluid bed processor such as a Glatt® Powder Coater-Granulator. Coating can be performed with a top spray process or a bottom spray process using, for example, a Wurster insert. Alternative core processing methods include, for example, granulation and extrusion-spheronization.

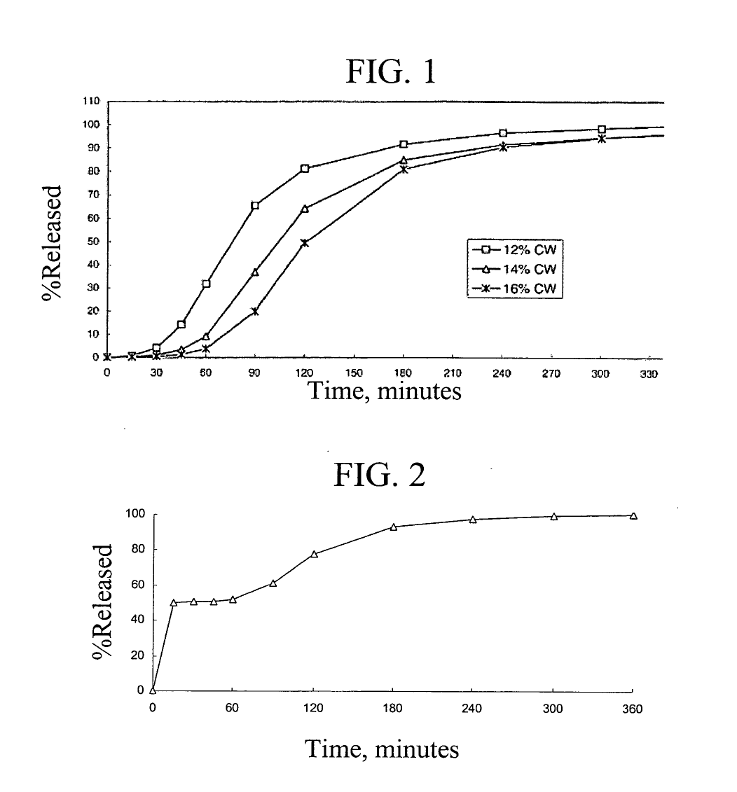

In order to achieve a desired dissolution profile, the sustained-release polymer coating weight may vary from about 8% to about 25% relative to the total weight of the coated pellet. Thus, by using cores of the same size and by increasing the thickness of the coating, the dissolution profile changes, even though the compositions of the core and the coating layer remain the same. Release profiles for sustained-release pellets having different coating weights are illustrated in FIG. 1 and described below.

In one embodiment, when measured in 900 mL 0.1 N HCl in a USP apparatus 2, a coated sustained-release pellet with about a 12 wt % sustained-release polymer coat weight exhibits the following dissolution profile: about 0% to about 25% of the total amphetamine salt is released after about 15 minutes, about 60% to about 100% of the total amphetamine salt is released after about 120 minutes, about 85% to about 100% of the total amphetamine salt is released after about 360 minutes.

In another embodiment, when measured in 900 mL 0.1 N HCl in a USP apparatus 2, a coated sustained-release particle with about a 14 wt % sustained-release polymer coat weight exhibits the following dissolution profile: about 0% to about 20% of the total amphetamine salt is released after about 15 minutes, about 45% to about 85% of the total amphetamine salt is released after about 120 minutes, about 75% to about 100% of the total amphetamine salt is released after about 360 minutes.

In another embodiment, when measured in 900 nL 0.1 N HCl in a USP apparatus 2, a coated sustained-release particle with about a 16 wt % sustained-release polymer coat weight exhibits the following dissolution profile: about 0% to about 15% of the total amphetamine salt is released after about 15 minutes, about 30% to about 60% of the total amphetamine salt is released after about 120 minutes, about 65% to about 100% of the total amphetamine salt is released after about 360 minutes;

The sustained-release coating may be dried before applying an optional outer coating. A color imparting agent may be added to the sustained-release coating composition or a rapidly dissolving seal coat containing color may be coated over the sustained-release coating layer provided that the seal coat is compatible with and does not affect the dissolution of the sustained-release coating layer.

The immediate-release and sustained-release pellets may be mixed and disposed in a capsule or formed into a tablet to form a sustained-release dosage form. The ratio of the immediate-release and sustained-release pellets in the dosage form may be about 5:95 to about 95:5; specifically about 25:75 to about 75:25; more specifically about 40:60 to about 60:40; most specifically about 50:50.

Suitable excipients to be added to a capsule formulation include, but are not limited to: fillers such as microcrystalline cellulose, soy polysaccharides, calcium phosphate dihydrate, calcium sulfate, lactose, sucrose, sorbitol, other inert fillers, and combinations comprising one or more of the foregoing fillers. In addition, there can be flow aids such as fumed silicon dioxide, silica gel, magnesium stearate, calcium stearate, other materials imparting flow to powders, and combinations comprising one or more of the foregoing flow aids. A lubricant can further be added such as, for example, polyethylene glycol, leucine, glyceryl behenate, magnesium stearate, calcium stearate, and combinations comprising one o more of the foregoing lubricants.

The immediate-release and sustained-release pellets may be made into tablets, for example, by first adding about 25 wt % to about 40 wt % of a solid pharmaceutically acceptable tablet excipient which will form a compressible mixture with the coated cores and which may be formed into a tablet without crushing the coated cores, and optionally an effective amount of a tablet disintegrating agent and a lubricant. The solid pharmaceutically acceptable tablet excipient may comprise, for example, lactose, dextrose, mannitol, calcium phosphate, microcrystalline cellulose, kaolin, powdered sucrose, or combinations comprising one or more of the foregoing excipients. The tablet disintegrant may comprise crospovidone, croscarmellose sodium, dry starch, sodium starch glycolate, and the like, and combinations comprising one or more of the foregoing disintegrants. Suitable lubricants include, for example, calcium stearate, glycerol behenate, magnesium stearate, mineral oil, polyethylene glycol, sodium stearyl fumarate, stearic acid, talc, vegetable oil, zinc stearate, and combinations comprising one or more of the foregoing lubricants. The immediate-release pellets and the sustained-release pellets may be added individually to a gelatin capsule.

The release profile of the sustained-release dosage forms disclosed herein is substantially independent of pH. Thus, the in vitro dissolution profiles substantially correspond to the following pattern when tested in various dissolution media at a pH range from about 1.0 to about 7.5. In addition to the sustained-release coating thickness, the ratio of immediate-release pellets to sustained-release pellets can be adjusted to give the desired release profile.

For example, when measured at pH 1.0 to 7.5, a sustained-release dosage form exhibits the following dissolution profile: about 35% to about 65% of the total amphetamine salts are released after about 30 minutes, about 55% to about 85% of the total amphetamine salts are released after about 120 minutes, and about 70% to about 100% of the total amphetamine salt are released after about 360 minutes.

For example, when measured in 900 mL 0.1 N HCl in a USP Apparatus 2, a sustained-release dosage form exhibits the following dissolution profile: about 35% to about 65% of the total amphetamine salts are released after about 30 minutes, about 55% to about 85% of the total amphetamine salts are released after about 120 minutes, and about 70% to about 100% of the total amphetamine salts are released after about 360 minutes.

In another example, when measured in 900 mL pH 4.5 buffer in a USP Apparatus 2, a sustained-release dosage form exhibits the following dissolution profile: about 35% to about 65% of the total amphetamine salts are released after about 30 minutes, about 55% to about 85% of the total amphetamine salts are released after about 120 minutes, and about 70% to about 100% of the total amphetamine salts are released after about 360 minutes.

In yet another example, when measured in 900 mL pH 6.8 buffer in a USP Apparatus 2, a sustained-release dosage form exhibits the following dissolution profile: about 35% to about 65% of the total amphetamine salts are released after about 30 minutes, about 55% to about 85% of the total amphetamine salts are released after about 120 minutes, and about 70% to about 100% of the total amphetamine salts are released after about 360 minutes.

In one embodiment, when measured under fasted conditions, the Cₘₐₓ for total amphetamine salts is about 45 ng/mL to about 78 ng/mL, and the Tₘₐₓ is about 4 hours to about 7 hours from administration. In another embodiment, when measured under fed conditions, the Cₘₐₓ for total amphetamine salts is about 40 ng/mL to about 70 ng/mL, and the Tₘₐₓ is about 4.5 hr to about 7.5 hr from administration.

The invention is further illustrated by the following non-limiting examples.

### EXAMPLE 1

### Formation of an Exemplary Sustained-release Dosage Form

The inert cores comprise 30 to 35 mesh sugar spheres. Sugars spheres were coated with a granulating suspension. The granulating suspension comprised 34,000 g of alcohol USP, 875 g hydroxypropyl cellulose, 1250 g of amphetamine aspartate monohydrate, 1250 g amphetamine sulfate, 1250 g dextroamphetamine saccharate, and 1250 g dextroamphetamine sulfate. The coating was performed in a fluid bed equipped with a Wurster processor. The operation parameters for the coating process were 300-400 cfm process air, 50° C. inlet air temperature, 0-10° C. inlet air dew point, 1-2 bar atomizing air pressure, 100-120 g/minute spray rate and a 19-22° C. product temperature. The coated sugar spheres produced, also referenced as coated cores thereof, were employed as immediate-release pellets and were also employed to produce sustained-release pellets.

The overall formulation for the coated cores is given in Table 1:

TABLE 1

| Component | Grams in 50,000 g batch |
|---|---|
| amphetamine aspartate monohydrate | 1250 |
| amphetamine sulfate | 1250 |
| dextroamphetamine saccharate | 1250 |
| dextroamphetamine sulfate | 1250 |
| Sugar sphere (30-35 mesh) | 44125 |
| Hydroxypropylcellulose | 875 |
| Alcohol* | 34,000 |

*alcohol was substantially removed during the coating process.

40,000 g of the cores produced above were coated with a coating composition comprising the materials shown in Table 2 to produce sustained-release pellets:

TABLE 2

| | Specific | |
|---|---|---|
| Component Type | component | Actual weight (g) |
| Sustained-release | Eudragit RL | 584 |
| polymer | | |
| Sustained-release | Eudragit RS | 5271 |
| polymer | | |
| Plasticizer | Triethyl citrate | 584 |
| Dispersing agent | Ethanol* | 49162 |
| Lubricant | Talc | 3620 |
| Total | | 59220 |

*ethanol was substantially removed during the coating process.

Coating was performed in a GPCG-15 fluid bed equipped with a Wurster processor. Processing conditions were: 500 cfm process air, 50° C. inlet air temperature, 18° C. inlet air dew point, 1.5 bar atomizing air pressure, 100 to 180 g/minute spray rate and a 35 to 40° C. product temperature. The coating weight was 20 wt % of the total weight of the coated sustained-release pellets.

The immediate-release pellets and the sustained-release pellets were then combined to produce a dosage form. The immediate-release pellets and the sustained-release pellets were added individually to a gelatin capsule. A suitable apparatus for production of a gelatin capsule is a commercial MG II Futura encapsulator. Capsules were produced having the compositions given in Table 3:

TABLE 3

| Dosage | Capsule | Immediate- | Sustained- | Individual |
|---|---|---|---|---|
| strength | shell size | release pellet fill | release pellet fill | capsule fill |
| (mg) | (#) | weight | weight (mg) | weight (mg) |
| 5 | 3 | 26 | 33 | 59 |
| 10 | 3 | 52 | 66 | 118 |
| 15 | 2 | 78 | 99 | 177 |
| 20 | 2 | 104 | 132 | 236 |
| 25 | 1 | 130 | 164 | 295 |
| 30 | 1 | 156 | 197 | 354 |

The dissolution profile of the 30 mg capsules was measured in 900 mL 0.1 N HCl in a USP apparatus 2. The dissolution profile exhibited is show in FIG. 2.

### EXAMPLE 2

### Formation of an Alternative Sustained-release Dosage Form

Cores comprising pharmaceutically active amphetamine salts were formed by preparing a granulating solution comprising 7020 g of alcohol USP and 180 g hydroxypropyl cellulose. The inert cores comprise 30 to 35 mesh sugar spheres. Coating was performed in a GPCG-5 fluid bed equipped with a Wurster processor. Processing conditions were: 200 cfm process air, 40° C. inlet air temperature, −4 to 3° C. inlet air dew point, 3 bar atomizing air pressure, 80 to 260 g/minute spray rate, 100-120 g/min binder suspension spray rate; and a 18-24° C. product temperature. A binder suspension was formed by mixing 7200 g of the granulating solution and 450 g of amphetamine aspartate monohydrate, 450 g amphetamine sulfate, 450 g dextroamphetamine saccharate, and 450 g dextroamphetamine sulfate. The overall formulation for the coated cores is given in Table 4:

TABLE 4

| Component | Grams in 16,000 g batch |
|---|---|
| amphetamine aspartate monohydrate | 450 |
| amphetamine sulfate | 450 |
| dextroamphetamine saccharate | 450 |
| dextroamphetamine sulfate | 450 |
| Sugar sphere (30-35 mesh) | 14020 |
| Hydroxypropylcellulose | 180 |

After coating, the coated cores were maintained in the rotor granulator to dry them. The coated sugar spheres produced were employed as immediate-release pellets and were also employed to produce sustained-release pellets.

3500 g of the cores produced above were coated with a sustained-release coating composition according to Table 5:

TABLE 5

| | Specific | |
|---|---|---|
| Component Type | component | Actual weight (g) |
| Sustained-release | Eudragit RL | 121 |
| polymer | | |
| Sustained-release | Eudragit RS | 482 |
| polymer | | |
| Plasticizer | Triethyl citrate | 60 |
| Dispersing agent | Ethanol* | 5066 |
| Lubricant | Talc | 330 |
| Total | | 6059 |

*ethanol was substantially removed during the coating process.

Coating was performed in a fluid bed equipped with a Wurster processor. Processing conditions were: 200 cfm process air, 40° C. inlet air temperature, 14 to 15° C. inlet air dew point, 1.5 bar atomizing air pressure, 20-80 g/min spray rate; and a 30-40° C. product temperature. The coating weight was about 20 wt % of the total weight of the coated pellets.

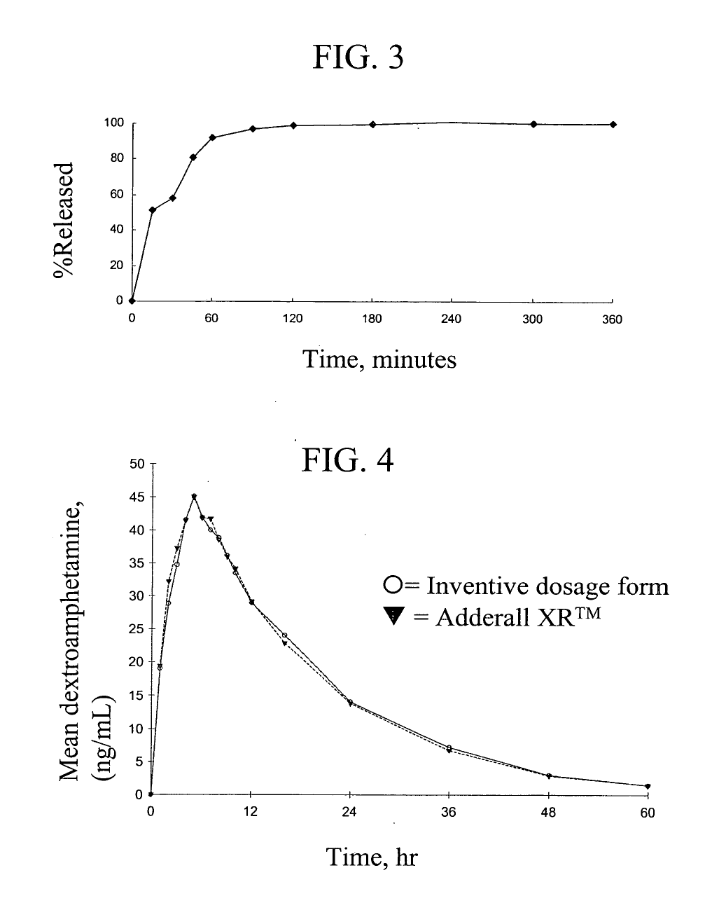

The immediate-release pellets and the sustained-release pellets were dosed into gelatin capsules using a double-dosing technique to give a 30 mg amphetamine capsule. The gelatin capsules contained 155 mg of the immediate-release pellets and 196 mg of the sustained-release pellets. The dissolution profile of the capsules was measured in 900 mL 0.1 N HCl in a USP apparatus 2. The dissolution profile is substantially as shown in FIG. 3.

### EXAMPLE 3

### Biostudy

A biostudy was conducted under fasted conditions. The study was designed as a randomized, single-dose two-way crossover to compare the rate and extent of absorption of a the amphetamine capsules of example 1 30 mg to Adderall XR™.

Twenty-six healthy adults participated in this comparison study and all of the subjects completed the study. Subjects received two separate drug administrations in assigned periods, one treatment per period, according to the randomization schedule. Dosing days were separated by a washout period of at least seven days. Blood samples were drawn prior to dosing (pre-dose) and at 1, 2, 3, 4, 5, 6, 7, 8, 9, 10, 12, 16, 24, 36, 48 and 60 hours post-dose. The samples were then analyzed.

The following pharmacokinetic parameters may be determined from the plasma concentration data:

The area under the plasma concentration versus time curve [AUC] may be calculated using the linear trapezoidal rule from the zero time point to the last quantifiable concentration.

The area under the plasma concentration versus time curve from zero to infinity [AUC_{0-INF}] may be calculated by adding Cₜ/Kₑₗₘ to AUC where Cₜ is the last quantifiable concentration and Kₑₗₘ is the elimination rate constant.

The maximum observed plasma concentration [Cₘₐₓ] may be obtained by inspection. The Cₘₐₓ may also be designated as CMAX.

The time to maximum plasma concentration [Tₘₐₓ] may be obtained by inspection. If the maximum plasma concentration occurs at more than one time point, the first may be chosen as Tₘₐₓ.

The terminal elimination rate constant [Kₑₗₘ] may be obtained from the slope of the line, fitted by linear least squares regression, through the terminal points of the log(base e) of the concentration versus time plot for these points.

The half-life [T_{1/2}] may be calculated by the equation T_{1/2}=0.693/Kₑₗₘ.

FIG. 4 shows the mean dextroamphetamine concentration versus time for inventive example 1 compared to Adderall XR™ when dosed under fasted conditions. Table 6 shows a comparison of the pharmacokinetic parameters for the inventive formulation compared to Adderall XR™ when dosed under fasted conditions. The release of dextroamphetamine was measured to calculate the pharmacokinetic parameters.

TABLE 6

| | Inventive Example 1 | | Adderall XR ™ | |
|---|---|---|---|---|
| | | Standard | | Standard |
| | Mean | deviation | Mean | deviation |
| Tₘₐₓ (hr) | 5.23 | 1.12 | 4.92 | 1.52 |
| Cₘₐₓ (ng/ml) | 47.4 | 11.9 | 47.9 | 6.74 |
| AUCₗₐₛₜ (hr * ng/ml) | 878.2 | 177.1 | 871.8 | 145.2 |
| AUC_{0-INF} (hr * ng/ml) | 904.5 | 189.7 | 896.5 | 156.9 |
| T_{1/2} (hr) | 10.46 | 2.13 | 10.52 | 2.10 |

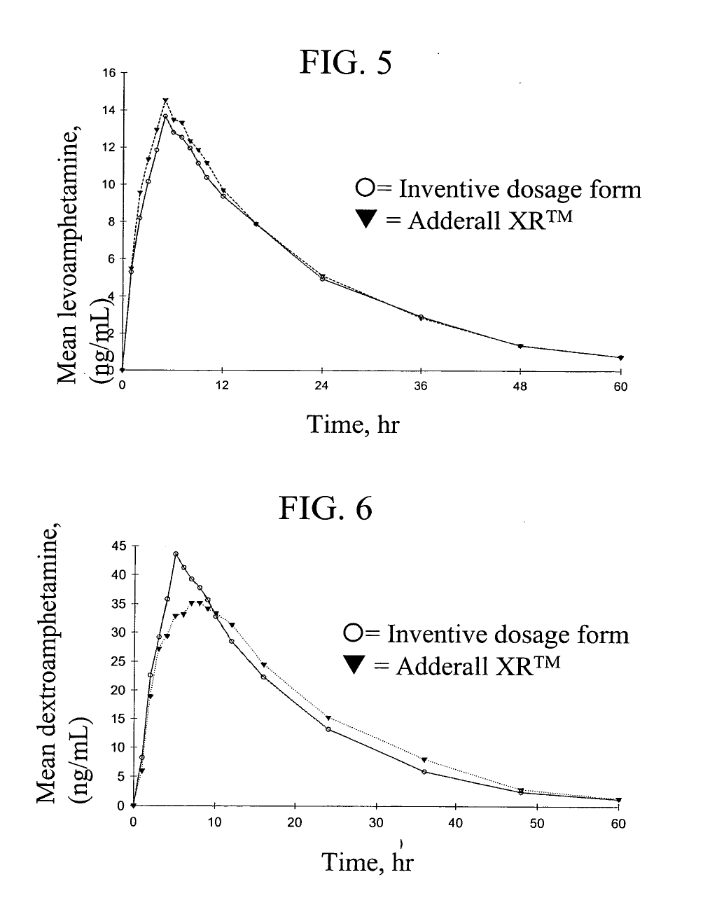

FIG. 5 shows the mean levoamphetamine concentration versus time for inventive dosage form 1 compared to Adderall XR™ when dosed under fasted conditions. Table 7 shows a comparison of the pharmacokinetic parameters for the inventive formulation compared to Adderall XR™ when dosed under fasted conditions. The release of levoamphetamine was measured to calculate the pharmacokinetic parameters.

TABLE 7

| | Inventive Example 1 | | Adderall XR ™ | |
|---|---|---|---|---|
| | | Standard | | Standard |
| | Mean | deviation | Mean | deviation |
| Tₘₐₓ (hr) | 5.46 | 1.36 | 4.96 | 4.51 |
| Cₘₐₓ (ng/ml) | 13.9 | 2.52 | 15.3 | 2.65 |
| AUC (hr * ng/ml) | 293.7 | 67.76 | 304.3 | 55.59 |
| AUC_{0-INF} (hr * ng/ml) | 310.3 | 77.82 | 320.1 | 63.32 |
| T_{1/2} (hr) | 12.72 | 3.26 | 12.45 | 3.00 |

A biostudy was also conducted under fed conditions. The study was designed as a randomized, single-way-way crossover to compare the rate and extent of absorption of a the amphetamine capsules of example 1 to 30 mg Adderall XR™.

Twenty-six healthy adults participated in this comparison study and the first twenty-four subjects completed the study. Subjects received two separate drug administrations in assigned periods, one treatment per period, according to the randomization schedule. Dosing days were separated by a washout period of at least seven days. Blood samples were drawn prior to dosing (pre-dose) and at 1, 2, 3, 4, 5, 6, 7, 8, 9, 10, 12, 16, 24, 36, 48 and 60 hours post-dose. The samples were then analyzed.

FIG. 6 shows the mean dextroamphetamine concentration versus time for inventive dosage form I compared to Adderall XR™ when dosed under fed conditions. Table 8 shows a comparison of the pharmacokinetic parameters for the inventive formulation compared to Adderall XR™ when dosed under fed conditions. The release of dextroamphetamine was measured to calculate the pharmacokinetic parameters.

TABLE 8

| | Inventive Example 1 | | Adderall XR ™ | |
|---|---|---|---|---|
| | | Standard | | Standard |
| | Mean | deviation | Mean | deviation |
| Tₘₐₓ (hr) | 5.54 | 1.14 | 8.17 | 2.60 |
| Cₘₐₓ (ng/ml) | 44.4 | 8.57 | 39.9 | 7.90 |
| AUCₗₐₛₜ (hr * Mng/ml) | 800.7 | 137.4 | 834.1 | 151.0 |
| AUC_{0-INF} (hr * ng/ml) | 821.7 | 143.1 | 854.1 | 158.6 |
| T_{1/2} (hr) | 10.42 | 1.49 | 10.11 | 1.22 |

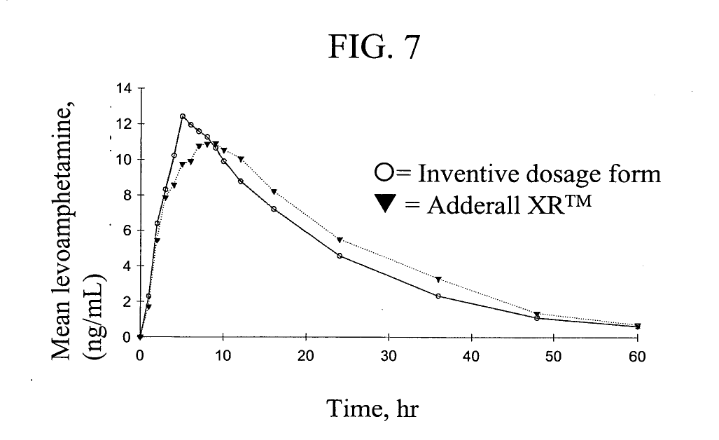

FIG. 7 shows the mean levoamphetamine concentration versus time for inventive dosage form 1 compared to Adderall XR™ when dosed under fed conditions. Table 9 shows a comparison of the pharmacokinetic parameters for the inventive formulation compared to Adderall XR™ with dosing in the fed conditions. The release of levoamphetamine was measured to calculate the pharmacokinetic parameters.

TABLE 9

| | Inventive Example 1 | | Adderall XR ™ | |
|---|---|---|---|---|
| | Standard | | | Standard |
| | Mean | deviation | Mean | deviation |
| Tₘₐₓ (hr) | 5.88 | 1.26 | 8.58 | 2.60 |
| Cₘₐₓ (ng/ml) | 12.7 | 2.08 | 12.6 | 2.68 |
| AUCₗₐₛₜ (hr * ng/ml) | 259.8 | 46.25 | 286.0 | 58.14 |
| AUC_{0-INF} (hr * ng/ml) | 272.4 | 50.66 | 298.8 | 64.46 |
| T_{1/2} (hr) | 12.42 | 2.25 | 12.05 | 1.89 |

For each of the fasted and fed conditions, the analysis of variance (ANOVA) and the Schuirmann's two one-sided t-test procedures at the 5% significance level were applied to the pharmacokinetic parameters obtained from the noncompartmental analysis Cₘₐₓ, Tₘₐₓ, AUCₗₐₛₜ, AUC_{0-INF} and T_{1/2}, and the long-transformed pharmacokinetic exposure parameters Cₘₐₓ, AUCₗₐₛₜ and AUC_{0-INF}. The 90% confidence interval for the difference between the means of the Inventive Example 1 capsules and Adderall XR™ was calculated. The lower and upper confidence intervals of the log-transformed parameters for the Inventive Example 1 capsules were within 80% -125% in comparison to that of Adderall XR™, however, the Inventive Example 1 exhibited a substantially no food effect when compared to Adderall XR™.

The use of the terms “a” and “an” and “the” and similar referents (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention as used herein, the terms wt %, weight percent, percent by weight, etc. are equivalent and interchangeable.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A sustained-release pharmaceutical dosage form comprising a plurality of particles comprising two or more pharmaceutically active amphetamine salts, wherein the dosage form, when administered under fed conditions, provides less than or equal to about a 30% increase or decrease in time to maximum blood plasma concentration of the pharmaceutically active amphetamine salts (Tₘₐₓ) as compared to the dosage form administered under fasted conditions.

2. The pharmaceutical dosage form of claim 1, wherein the dosage form, when administered under fed conditions, provides less than or equal to about a 15% increase or decrease in time to maximum blood plasma concentration of the pharmaceutically active amphetamine salts (Tₘₐₓ) as compared to the dosage form administered under fasted conditions.

3. The pharmaceutical dosage form of claim 1, wherein the dosage form, when administered under fed conditions, provides less than or equal to about a 10% increase or decrease in time to maximum blood plasma concentration of the pharmaceutically active amphetamine salts (Tₘₐₓ) as compared to the dosage form administered under fasted conditions.

4. The pharmaceutical dosage form of claim 1, wherein the Tₘₐₓ is about 4 hours to about 7 hours from administration under fasted conditions and about 4.5 hours to about 7.5 hours from administration under fed conditions.

5. A sustained-release pharmaceutical dosage form comprising a plurality of particles comprising two or more pharmaceutically active amphetamine salts, wherein the dosage form, when administered under fed conditions, provides less than or equal to about a 15% increase or decrease in peak exposure of the pharmaceutically active amphetamine salts (Cₘₐₓ) as compared to the dosage form administered under fasted conditions.

6. The pharmaceutical dosage form of claim 5, wherein the dosage form, when administered under fed conditions, provides less than or equal to about a 12% increase or decrease in peak exposure of the pharmaceutically active amphetamine salts (Cₘₐₓ) as compared to the dosage form administered in fasted conditions.

7. The pharmaceutical dosage form of claim 5, wherein the dosage form, when administered under fed conditions, provides less than or equal to about a 10% increase or decrease in peak exposure of the pharmaceutically active amphetamine salts (Cₘₐₓ) as compared to the dosage form administered under fasted conditions.

8. The pharmaceutical dosage form of claim 5, wherein the Cₘₐₓ is about 45 ng/mL to about 78 ng/mL under fasted conditions and about 40 ng/mL to about 70 ng/mL under fed conditions.

9. The pharmaceutical dosage form of claim 1 or 5, having a confidence interval of about 80% to 125% of the log-transformed parameter in comparison to the same parameter of ADDERAL XR™, wherein the parameter is selected from the group consisting of Cₘₐₓ, AUCₗₐₛₜ and AUC_{0-INF}.

10. A sustained-release pharmaceutical dosage form for administration of two or more pharmaceutically active amphetamine salts to a human, wherein the dosage form comprises a plurality of particles comprising:
a first population of immediate-release particles; and
a second population of sustained-release particles, the sustained-release particles comprising one or more sustained-release polymers, wherein the sustained-release particles comprise no enteric release polymers;
wherein the first and second populations comprise two or more pharmaceutically active amphetamine salts.

11. The pharmaceutical dosage form of claim 10, wherein the pharmaceutically active amphetamine salts comprise two or more of dextroamphetamine sulfate, dextroamphetamine saccharate, amphetamine aspartate monohydrate, and amphetamine sulfate.

12. The pharmaceutical dosage form of claim 10, wherein the plurality of particles are pellets.

13. The pharmaceutical dosage form of claim 12, wherein the immediate-release pellets comprise about 50 wt % to about 90 wt % of an inert core, about 0.001 wt % to about 4 wt % of a first binder, and about 5 wt % to about 15 wt % of the pharmaceutically active amphetamine salts, all based on the total weight of the immediate-release pellets.

14. The pharmaceutical dosage form of claim 12, wherein the sustained-release pellets comprise an inert core; a first coating comprising a binder and the pharmaceutically active amphetamine salts disposed on the inert core; and a second coating comprising the sustained-release polymer disposed on the first coating.

15. The pharmaceutical dosage form of claim 14, wherein the sustained-release pellets comprise about 50 wt % to about 90 wt % of the inert core, about 0.001 wt % to about 4 wt % of the binder, about 5 wt % to about 15 wt % of the pharmaceutically active amphetamine salts, and about 10 wt % to about 40 wt % of the sustained-release polymer, all based on the total weight of the sustained-release pellets.

16. The pharmaceutical dosage form of claim 14, wherein the binder comprises hydroxypropyl cellulose.

17. The pharmaceutical dosage form of claim 14, wherein the sustained release polymer comprises cellulose acetate, cellulose acetate butyrate, ethyl cellulose acetate propionate, ethyl cellulose, a fatty acid ester, a wax, zein, a copolymer of acrylate and methacrylate with quaternary ammonium groups, a cellulose acetate latex, or combination of one or more of the foregoing sustained-release polymers.

18. The pharmaceutical dosage form of claim 17, wherein the sustained-release polymer comprises a copolymer synthesized from acrylic and methacrylic acid esters with quaternary ammonium groups.

19. The pharmaceutical dosage form of claim 18, wherein the copolymer synthesized from acrylic and methacrylic acid esters with quaternary ammonium groups comprises about 80:20 to about 90:10 of a first copolymer that is permeable to water to a second copolymer that is less permeable to water.

20. The pharmaceutical dosage form of claim 10, exhibiting a dissolution profile in 900 mL 0.1 N HCl in USP apparatus 2 such that:
about 35 wt % to about 65 wt % of the total amphetamine salts are released after about 30 minutes;
about 55 wt % to about 85 wt % of the total amphetamine salts are released after about 120 minutes; and
about 70 wt % to about 100 wt % of the total amphetamine salts are released after about 360 minutes.

21. A method of treating a human comprising administering a pharmaceutically effective amount of the dosage forms of claim 1 or 4 or 10 to a human in need of treatment for Attention-Deficit/Hyperactivity Disorder.

22. The method of claim 21, wherein the human is in need of treatment for Attention-Deficit/Hyperactivity Disorder and is a child of age 3 to 10.
